(19) United States
(12) Patent Application Publication
(10) Pub. No.: US 2016/0017319 A1
(43) Pub. Date: Jan. 21, 2016

(54) Method of screening cell clones

(71) Applicants: Audrey NOMMAY, (US); Burkhard WILMS, (US)

(72) Inventors: Audrey NOMMAY, Blotzheim (FR); Burkhard WILMS, Weil am Rhein (DE)

(21) Appl. No.: 14/773,966

(22) PCT Filed: Mar. 10, 2014
(86) PCT No.: PCT/IB2014/059584

§ 371 Date: Sep. 9, 2015

Related U.S. Application Data
(60) Provisional application No. 61/776,176, filed on Mar. 11, 2013.

(51) Int. Cl. C12N 15/10

(52) U.S. Cl. CPC C12N 15/1079

(57) ABSTRACT

A method of screening cell clones expressing a high yield of a polypeptide of interest is provided. The method employs the consecutive use of fluorescence activated cell sorting followed by colony picking based selection of cell clones with high expression rates and high proliferation rates. Furthermore, the invention pertains to a method of producing a polypeptide of interest using cells obtained by the described screening method.

### FIELD OF THE INVENTION

The present disclosure concerns the field of cell culture technology. It pertains to a method of screening cell clones expressing a high yield of a polypeptide of interest. Furthermore, the disclosure pertains to a method of producing a polypeptide of interest using cells obtained by the described screening method.

### BACKGROUND OF THE INVENTION

The market for biopharmaceuticals continues to grow at a high rate as biopharmaceuticals become more and more important for today's medicine. Currently, an increasing number of biopharmaceuticals is produced in eukaryotic cells such as in particular mammalian cells. Successful and high yield production of biopharmaceuticals in eukaryotic cells is thus crucial and depends on the characteristics of the recombinant monoclonal cell line used in the process. In addition, the time to generate such a mammalian cell line producing a therapeutic protein of interest is an essential part of the time needed to bring any biopharmaceutical in the clinic. Furthermore, also considering the enormous production costs for biopharmaceuticals it is important to have high expressing eukaryotic cell lines. Thus, there is a need to develop methods to screen high producing cell lines as fast as possible while maintaining the quality of the cell lines resulting from such a screening process, in particular with regard to their long term productivity.

Generation of mammalian production cells generally requires a cloning step to ensure that the population of cells grown in a bioreactor is genetically as homogenous as possible. Current methods for selecting stable, high-secreting clones are for example described in Burke and Mann (Bioprocess International, May 2006, pages 48 to 51: Rapid isolation of monoclonal antibody producing cell lines). Current methods in use are limiting dilution, FACS selection and the use of a clone picking robots.

The principle behind the method of limited dilution is that cells derived from a mixed population are separated from each other and further grown as single, isolated cells in culture. The progeny from the cell divisions of each isolated cell will be a clone of identical cells. If the original cell was secreting antibodies, then all of the members of the clone should secrete antibodies that are identical. Once an antibody-secreting clone is established, it can be expanded to allow for the production of large amounts of the antibody. However, the technique of cloning by limited dilution is laborious and time consuming.

Other common selection processes that allow screening a large number of cells in short time involve the use of a flow cytometer. Flow cytometers can be used to detect and also sort fluorescently labelled cells in a flowing stream of liquid. Fluorescence activated cell sorting (FACS) allows the sorting of high expressing cells in bulk according to a predefined threshold criteria, or the sorting of individual cells fulfilling the desired expression criteria into multi-well plates, each cell into a separate well. The selection speed for FACS can reach more than 60 million cells per hour. FACS based systems are also suitable for automation. Different FACS based selection systems that allow the selection of high expressing cells are for example disclosed in WO 2003/099996, WO 2003/014361, WO 2005/073375, WO 2010/022961 and WO 2007/131774.

As alternative to flow cytometry based selection systems clone picking robots are used to automatically select and transfer mammalian cellular colonies (see for example Burke and Mann 2006; Caron et al, BMC Biotechnology 9(42):1-11, 2009; Serpieri et al, Mol. Biotechnol. 45:218-225, 2010; EP 2 151 689, EP 1 752 771, EP 1 754 537). Clone picking robots such as the ClonePix FL (GENETIX) consist of a cabinet, a built in imaging device, various sources of illumination including white light and fluorescent light, suitable light detectors collecting the emitted light and a clone picking head to be moved over the precise position of the colony of interest, pick the colony and transfer it for further cultivation to an appropriate culture plate. By tagging high producing colonies with a fluorescent marker, colonies can be sorted by the dual parameters of colony size and expression rate of the molecule of interest. The ClonePix FL colony picking robot reaches a picking speed of up to 400 colonies per hour. An important difference to the principle of cell sorting by FACS is that a colony picking robot by virtue does not sort single cells but sorts colonies. A cell colony consists of many cells, wherein all cells derive from the same initial colony founding cell.

For biopharmaceutical production efficiency's sake in particular on industrial scale, a tremendous effort is put into the clone selection process, with the goal to identify high producing clones with a high growth rate in a short amount of time. Even when using highly stringent selection systems that favour survival of high expressing cells under the used selection conditions, finding a suitable production clone within the surviving population which combines a high expression rate with good growth characteristics is all about numbers. The more clones one can screen with the available time and resource, the greater the chance of finding clones that combine a high productivity with a fast growth rate. Clone selection in industry is required to be fast and reliable and must enable the parallel selection of thousands of clones from many projects. To achieve this challenging goal, automated high throughput screening platforms have been setup. At each position of the multi-step selection process, special robotic equipment is integrated to fulfil a certain task. As such, the screening process for suitable clones can be set up as semi-automated process, including the steps of host cell transfection, clone selection and cloning phase (or clone propagation or expansion phase).

As described above, for clone selection on large scale, in the prior art either FACS sorting or clone picking is used. Sorted cells or colonies are often transferred to multi-well plates for further handling. Clone propagation is preferably assisted by automated cell handling systems, also referred to as cloning robots, in order to allow the handling of large number of clones. The use of cloning robots is standard in industry. Such cloning robots consist of a cabinet to cultivate many multi-well plates in parallel and are equipped with internal devices such as a spectrophotometer and a liquid handling system enabling in the multi-well plate format the pre-selection of clones with high growth and high production rate. In such robots the amount of protein is determined, thereby allowing to select based on the protein titer in the cell culture medium on a small scale cells that express a high amount of the protein of interest. Clones pre-selected with the aid of a cloning robot can then be analyzed in more detail in further downstream analysis. Thus, when including a cloning robot in the screening platform, the throughput rate of the whole screening platform ultimately depends on the working efficiency of its finishing element, the cloning robot. Because the cloning robot is loaded with multi-well plates that are passed over from the clone selection step, the working efficiency of the cloning robot is directly linked to the efficiency and throughput rate of the upstream clone selection process. The use of FACS selection in combination with a cloning robot is e. g. described in WO 2008/031873.

Although FACS sorting and clone picking have been used extensively in industry for clone selection, both methods intrinsically have drawbacks and none has delivered fully satisfactory results.

In practical terms, single isolated cells derived from clone selection by FACS often have a poor growth rate, need a long cultivation-time to grow from a single cell to a dense culture. Furthermore, some cells might not grow at all. It is not possible to evaluate the growth characteristics of FACS-sorted cells until they have been cultured for proliferation. Therefore, non-growing cells are just identified on the cloning robot level. For example, it has been observed that only 30% of FACS-selected CHO cells ultimately grow to appropriate culture densities in multi-well culture plates that are cultured in the cloning robot. Thus, most of the wells of the plates are left blank. Hence, the cloning robot receives multi-well culture plates from the FACS sorting process wherein, however, a high number of wells (in case of CHO cells often 70%) represent a loss because cells do not grow to dense cultures. As incubator space in the cloning robot is both precious and limited to a certain number of multi-well plates, the working efficiency of cloning robots loaded with multi-well plates with numerous “empty” wells is sub-optimal and a waste of cloning robot capacity. Furthermore, working with multi-well plates in which wells remain empty increases the costs for the screening process. Furthermore, less projects can be handled in parallel. In addition, those clones that do proliferate often require a long time to grow to dense cultures—time during which the cloning robot is occupied and cannot be used for other projects. Thus, the poor cloning efficiency (often less than 50% or even less than 30%) of flow cytometry based selection processes is a major drawback of this system. The cloning efficiency can vary from cell to cell and also depends on the membrane stability of the cell.

In contrast to flow cytometry based approaches, clone picking based approaches have a high cloning efficiency. Thus, significantly less “empty” wells are present in the multi-well plates that are passed over to the cloning robot. Furthermore, a clone picking robot does not pick single cells but cell colonies. Clone colonies grow faster to dense cultures than single cells. The main disadvantage of this method is the slow speed of the colony picker (400 colony clones/hour, compared to FACS>10⁷ cellular clones/hour). The throughput of the colony picker is further limited to the density of individual clones which can be plated in semi-solid medium in the 1-well plate. As a consequence, automated clone picking is time consuming and much slower than FACS sorting and less cells can be screened. Thus, the main drawback of a colony picking based selection is the lack of high throughput clone screening. This limits the chances to find the rare ultra-high producing cell clones that have good growth characteristics in the population of successfully transfected cells.

In summary, all state of the art clone selection methods, such as limiting dilution, FACS sorting and colony picking have advantages and drawbacks, and neither method provides the optimal solution to achieve a satisfying high throughput rate for screening high producing cells with fast growth rate. There is thus a need for improved clone selection methods that are suitable for high throughput applications and can be performed by automated laboratory equipment and thus meet the industry needs.

It is an object of the invention to overcome at least one drawback of the prior art methods. In particular, it is an object of the present invention to provide an improved method for selecting high producing clones with high growth rate.

### SUMMARY OF THE INVENTION

The present inventors found that a multiple-step screening method wherein a flow cytometry based selection is followed by a colony picking based selection greatly improves the cloning efficiency and therefore increases the number of high expressing cell clones that is obtained from the screening process. It was found that this specific serial combination of techniques that are used in the prior art as alternative selection methods results in a significantly higher throughput rate and a higher cloning efficiency. The flow cytometry based selection step allows to screen a large number of cells within a short time frame for their expression characteristics, thereby allowing to select a population of cells which express the polypeptide of interest with high yield. The subsequent colony picking based selection advantageously starts from this population of high expressing cells. Thus, the high cloning efficiency of the colony picking based selection is advantageously focused on the high producing cells that were identified in the flow cytometry based selection. Thus, the serial combination of these two selection strategies enables to keep the selectivity and high throughput of flow cytometry based selection for selecting high expressing cells, while focussing the high cloning efficiency of a colony picking based selection on these flow cytometry selected, high expressing cells. As a result of this specific serial combination of selection steps a higher amount of cell clones is provided which in particular combine a high expression rate with good growth characteristics. More cells can be screened in order to identify clones having the desired characteristics. Furthermore, as high producing cell clones which additionally show good characteristics in the clone picking step are cultivated to provide cell clones, more cell clones having the optimal expression and growth characteristics can be obtained from such a screening process and/or more projects can be handled in parallel. Therefore, compared to prior art screening methods, resources are more effectively used and the number of low producing host cells or cells having unfavourable growth characteristics is significantly reduced. Thereby, also the costs for screening can be significantly reduced (up to approximately 50% of the screening costs can be saved). Thus, the serial combination of flow cytometry and clone picking based selection as taught herein greatly reduces the efforts and costs needed to identify cell clones with a desirous combination of expression rate, cell viability and growth rate, as compared to methods described in the state of the art. These are important advantages in industry for large scale production of recombinant polypeptides of interest. Therefore, the present invention makes an important contribution to the prior art. The multiple-step screening method is particularly suitable for use with a cultivation and selection using a cloning robot after the colony picking step, as the number of high expressing clones which can be handled in parallel by the cloning robot is increased, thereby getting closer to the maximal cloning robot usage efficiency.

According to a first aspect, a screening method is provided for selecting at least one cell clone with desired colony characteristics expressing a polypeptide of interest, the method comprising
a) providing a plurality of eukaryotic host cells comprising a heterologous nucleic acid comprising a polynucleotide encoding the polypeptide of interest;
b) cultivating the eukaryotic host cells;
c) performing a first, flow cytometry based selection, comprising
   selecting a plurality of eukaryotic host cells expressing the polypeptide of interest with desired yield using flow cytometry;
d) performing a second, colony picking based selection, comprising
   obtaining single cell colonies in a medium which prevents the migration of the cells from a plurality of eukaryotic host cells selected in stage c);
   detecting within the obtained cell colonies one or more cell colonies having desired colony characteristics;
   picking one or more cell colonies having the desired colony characteristics;
e) cultivating the picked cell colonies to provide cell clones.

According to a second aspect, a method is provided for producing a polypeptide of interest, comprising
a) culturing a cell clone selected according to the method of the first aspect under conditions that allow for the expression of the polypeptide of interest; and
b) isolating the polypeptide of interest from the cell culture medium and/or from the cells.

Other objects, features, advantages and aspects of the present application will become apparent to those skilled in the art from the following description and appended claims. It should be understood, however, that the following description, appended claims, and specific examples, while indicating preferred embodiments of the application, are given by way of illustration only. Various changes and modifications within the spirit and scope of the disclosed invention will become readily apparent to those skilled in the art from reading the following.

### DETAILED DESCRIPTION OF THE INVENTION

According to a first aspect, a screening method is provided for selecting at least one cell clone with desired colony characteristics expressing a polypeptide of interest, the method comprising
a) providing a plurality of eukaryotic host cells comprising a heterologous nucleic acid comprising a polynucleotide encoding the polypeptide of interest;
b) cultivating the eukaryotic host cells;
c) performing a first, flow cytometry based selection, comprising
   selecting a plurality of eukaryotic host cells expressing the polypeptide of interest with desired yield using flow cytometry;
d) performing a second, colony picking based selection, comprising
   obtaining single cell colonies in a medium which prevents the migration of the cells from a plurality of eukaryotic host cells selected in stage c);
   detecting within the obtained cell colonies one or more cell colonies having desired colony characteristics;
   picking one or more cell colonies having the desired colony characteristics;
e) cultivating the picked cell colonies to provide cell clones.

The advantages of the screening method were already briefly described above in the summary of the invention. The present invention provides an improved cell cloning and selection procedure. Important is the specific serial use of the respective screening technologies to achieve the advantages that are described herein. High producing cell clones are identified by performing an enrichment of high producing cells using a flow cytometer, wherein afterwards, a clone picking step is performed in order to pick the best producing cell colonies based on their growth rate. This step is performed using the already flow cytometry, preferably FACS, enriched cells, which thus were already identified as being high producers. Therefore, the colonies that are grown on the level of clone picking already have favourable characteristics regarding their expression rate. Therefore, the lower throughput efficiency of the clone picking system is compensated by the preceding flow cytometry based selection, as only high producing cells are plated and thus analysed at this stage. Furthermore, even though in the present invention an additional selection stage is included, it was found that the overall screening time is not necessarily increased as is shown in the examples. The individual stages as well as suitable and preferred embodiments will now be explained in detail.

### Stage A—Generation of Host Cells Comprising a Heterologous Nucleic Acid

In stage a), a plurality of eukaryotic host cells is provided which comprise a heterologous nucleic acid comprising a polynucleotide encoding the polypeptide of interest.

A “heterologous nucleic acid” in particular refers to a polynucleotide sequence that has been introduced into a host cell e.g. by the use of recombinant techniques such as transfection. A “polynucleotide” in particular refers to a polymer of nucleotides which are usually linked from one deoxyribose or ribose to another and refers to DNA as well as RNA, depending on the context. The term “polynucleotide” does not comprise any size restrictions. The host cell may or may not comprise an endogenous polynucleotide corresponding to, respectively being identical to the polynucleotide encoding the polypeptide of interest. The heterologous nucleic acid may consist of or may comprise an expression cassette. Preferably, the heterologous nucleic acid is an expression vector which comprises an expression cassette comprising the polynucleotide encoding the polypeptide of interest. Introduction into the cells may be achieved e.g. by transfecting a suitable expression vector comprising the polynucleotide encoding the polypeptide of interest into the host cells. The expression vector may integrate into the genome of the host cell (stable transfection). In case the heterologous nucleic acid is not inserted into the genome, the heterologous nucleic acid can be lost at the later stage e.g. when the cells undergo mitosis (transient transfection). Vectors might also be maintained in the host cell without integrating into the genome, e.g. by episomal replication. Stable transfection is preferred for generating high expressing cell clones that are suitable for producing a polypeptide of interest on industrial scale. There are several appropriate methods known in the prior art for introducing a heterologous nucleic acid such as an expression vector into eukaryotic host cells. Respective methods include but are not limited to calcium phosphate transfection, electroporation, lipofection, biolistic- and polymer-mediated genes transfer and the like. Besides traditional random integration based methods also recombination mediated approaches can be used to transfer the heterologous nucleic acid into the host cell genome. As respective methods are well known in the prior art, they do not need any detailed description here. Non-limiting embodiments of suitable vector designs will also be described subsequently.

The heterologous nucleic acid preferably is an expression vector. Expression vectors used for expressing recombinant products of interest usually contain transcriptional control elements suitable to drive transcription such as e.g. promoters, enhancers, polyadenylation signals, transcription pausing or termination signals usually as element of an expression cassette. If the desired product is a polypeptide, suitable translational control elements are preferably included in the vector, such as e.g. 5′ untranslated regions leading to 5′ cap structures suitable for recruiting ribosomes and stop codons to terminate the translation process. In particular, the polynucleotide encoding the product of interest as well as polynucleotides serving as the selectable marker or reporter genes can be transcribed under the control of transcription elements present in appropriate promoters. The resultant transcripts harbour functional translation elements that facilitate protein expression (i.e. translation) and proper translation termination. A functional expression unit, capable of properly driving the expression of an incorporated polynucleotide is also referred to as an “expression cassette” herein. As described, an expression cassette may comprise promoters, ribosome binding sites, polyadenylation signals, enhancers and other control elements which regulate transcription of a gene or translation of an mRNA. The exact structure of expression cassette may vary as a function of the species or cell type, but may comprise 5′-untranscribed and 5′- and 3′-untranslated sequences which are involved in initiation of transcription and translation, respectively, such as TATA box, capping sequence, CAAT sequence, and the like. More specifically, 5′-untranscribed expression control sequences comprise a promoter region which includes a promoter sequence for transcriptional control of the operatively connected nucleic acid. Expression cassettes may also comprise enhancer sequences or upstream activator sequences. The polynucleotide(s) encoding the product of interest and the polynucleotides encoding the selectable marker(s) and/or reporters as described herein are preferably comprised in an expression cassette. Several embodiments are suitable, for example each of said polynucleotide(s) can be comprised in a separate expression cassette. However, it is also within the scope of the present invention that at least two of the respective polynucleotides are comprised in one expression cassette. According to one embodiment, at least one internal ribosomal entry site (IRES) element is functionally located between the polynucleotides that are expressed from the same expression cassette. Thereby, it is ensured that separate translation products are obtained from said transcript. Respective IRES based expression technologies are well known in the prior art and thus need no further description here.

As described, the expression vector may comprise at least one promoter and/or promoter/enhancer element as element of an expression cassette. Although the physical boundaries between these two control elements are not always clear, the term “promoter” usually refers to a site on the nucleic acid molecule to which an RNA polymerase and/or any associated factors binds and at which transcription is initiated. Enhancers potentiate promoter activity, temporally as well as spatially. Many promoters are transcriptionally active in a wide range of cell types. Promoters can be divided in two classes, those that function constitutively and those that are regulated by induction or derepression. Both are suitable in conjunction with the teachings of the present disclosure. Promoters used for high-level production of proteins in mammalian cells should be strong and preferably active in a wide range of cell types. Strong constitutive promoters which drive expression in many cell types include but are not limited to the adenovirus major late promoter, the human cytomegalovirus immediate early promoter, the SV40 and Rous Sarcoma virus promoter, and the murine 3-phosphoglycerate kinase promoter, EF1a. According to one embodiment, the promoter and/or enhancer is either obtained from CMV and/or SV40. The transcription promoters can be selected from the group consisting of an SV40 promoter, a CMV promoter, an EF1alpha promoter, a RSV promoter, a BROAD3 promoter, a murine rosa 26 promoter, a pCEFL promoter and a β-actin promoter.

Furthermore, an expression cassette may comprise at least one intron. This embodiment is particularly suitable if a mammalian host cell is used for expression. Most genes from higher eukaryotes contain introns which are removed during RNA processing. Respective constructs are expressed more efficiently in transgenic systems than identical constructs lacking introns. Usually, introns are placed at the 5′ end of the open reading frame but may also be placed at the 3′ end. Accordingly, an intron may be comprised in the expression cassette(s) to increase the expression rate of the polypeptide of interest. Said intron may be located between the promoter and or promoter/enhancer element(s) and the 5′ end of the open reading frame of the polynucleotide to be expressed. Several suitable introns are known in the state of the art that can be used in conjunction with the present disclosure. According to one embodiment, the intron used in the expression cassettes for expressing the product of interest, is a synthetic intron such as the SIS or the RK intron. The RK intron is a strong synthetic intron which is preferably placed before the ATG start codon of the gene of interest. The RK intron consists of the intron donor splice site of the CMV promoter and the acceptor splice site of the mouse IgG Heavy chain variable region (see e.g. Eaton et al., 1986, Biochemistry 25, 8343-8347; Neuberger et al., 1983, EMBO J. 2(8), 1373-1378; it can be obtained from the pRK-5 vector (BD PharMingen)).

In preferred embodiments, the expression vector comprising the polynucleotide encoding the polypeptide of interest additionally comprises at least one polynucleotide encoding a selectable marker or a reporter. A “selectable marker” allows under appropriate selective culture conditions the selection of host cells expressing said selectable marker. A selectable marker provides the carrier of said marker under selective conditions with a survival and/or growth advantage. Thereby, host cells successfully transfected with the expression vector can be selected. Typically, a selectable marker gene will confer resistance to a selection agent such as a drug, e.g. an antibiotic, or compensate for a metabolic or catabolic defect in the host cell. It may be a positive or negative selection marker. For selecting successfully transfected host cells the culture medium used for culturing the host cells comprises a selection agent that allows selection for the selectable marker used. In other embodiments, the selection marker enables the host cell to survive and proliferate in the absence of a compound which is essential for survival and/or proliferation of the host cells lacking the selection marker. By cultivating the host cells in a medium which does not comprise the essential compound in a concentration high enough for survival and/or proliferation of the host cell, only host cells expressing the selection marker can survive and/or proliferate. According to one embodiment, the selectable marker is a drug resistance marker encoding a protein that confers resistance to selection conditions involving said drug. According to one embodiment, at least one selectable marker is used which confers resistance against one or more antibiotic agents. Selectable marker genes commonly used with eukaryotic cells include the genes for aminoglycoside phosphotransferase (APH), hygromycin phosphotransferase (hyg), dihydrofolate reductase (DHFR), thymidine kinase (tk), glutamine synthetase, asparagine synthetase, and genes encoding resistance to neomycin (G418), puromycin, hygromycin, zeocin, ouabain, blasticidin, histidinol D, bleomycin, phleomycin and mycophenolic acid. Also suitable is the use of the folate receptor as selectable marker (see WO 2009/080759). Suitable other examples for selectable markers are also well-known in the art. A variety of marker genes have been described (see, e.g., WO 1992/08796, WO 1994/28143, WO 2004/081167, WO 2009/080759, WO 2010/097240.

The selectable marker may according to one embodiment be an amplifiable selectable marker. An amplifiable selectable marker allows the selection of vector containing host cells and may promote gene amplification of said vector in the host cells. Thereby, the polynucleotide encoding the polypeptide of interest which is introduced by the vector into the host cell can be amplified into multiple copies in the host cell, providing for a higher and more stable expression. An “amplifiable selectable marker gene” has the properties of a selectable marker as defined above, but additionally can be amplified under appropriate conditions. The amplifiable selectable marker gene usually encodes an enzyme which is required for growth of eukaryotic cells under those conditions. For example, the amplifiable selectable marker gene may encode DHFR (dihydrofolate reductase), a suitable selective agent is e.g. methotrexate. Another example is the glutamine synthetase (GS) system. Further exemplary amplifiable selectable markers are also known in the art (see, e.g., WO 01/04306).

As used herein, a “selection medium” is a cell culture medium useful for the selection of host cells. It may include a selection agent such as a toxic drug which allows to identify successfully transfected host cells which have incorporated the expression vector.

A “reporter” allows the identification of a cell comprising said reporter based on the reporting characteristics (e.g. fluorescence). Reporter genes usually do not provide the host cells with a survival advantage. However, the expression of the reporter can be used to differentiate between cells expressing the reporter and those which do not. Therefore, also a reporter gene enables the selection of successfully transfected host cells. Suitable reporters include but are not limited to as e.g. green fluorescence protein (GFP), YFP, CFP and luciferase. According to one embodiment, the reporter used has characteristics that enable the selection by flow cytometry.

The expression vector may also comprise more than one selectable marker and/or reporter. Furthermore, the one or more selectable markers and/or the one or more reporters may also be provided on a separate expression vector which is then co-transfected with the expression vector which encodes the polypeptide of interest. Such co-transfection strategies also enable selection as is well-known in the prior art.

Preferably, the eukaryotic host cell is a mammalian cell. Said mammalian cell is preferably selected from the group consisting of a rodent cell, a human cell and a monkey cell. Particularly preferred is a rodent cell, which preferably is selected from the group consisting of a CHO cell, a BHK cell, a NS0 cell, a mouse 3T3 fibroblast cell, and a SP2/0 cell. A most particularly preferred rodent cell is a CHO cell. Also suitable is a human cell, which, may be e.g. selected from the group consisting of a HEK293 cell, a MCF-7 cell, a PerC6 cell, a CAP cell and a HeLa cell. Also suitable is a monkey cell, which, e.g. may be selected from the group consisting of a COS-1, a COS-7 cell and a Vero cell.

### Stage B—Cultivation

In stage b) the eukaryotic host cells are cultivated. One or more selection steps can be performed in stage b), e.g. in order to identify successfully transfected cells. The suitable selection strategies depend on the design of the expression vector that is used for introducing the polynucleotide encoding the polypeptide of interest and in particular depends on the used selection marker(s) and/or reporter(s).

When using an expression vector comprising a polynucleotide encoding a selectable marker, the host cells may be cultivated under conditions providing a selection pressure to identify successfully transfected host cells. According to one embodiment, host cells which were not successfully transfected and hence, do not express the selection marker(s) (and accordingly the protein of interest) or only express them with low yield cannot proliferate or die under the cultivation conditions. In contrast, host cells which are successfully transfected with the expression vector and which express the selection marker(s) with sufficient yield are resistant to or less affected by the selection pressure and can normally proliferate, thereby outgrowing the host cells which are not successfully transfected. The selection pressure is preferably provided by the use of a selection medium for cultivation of the eukaryotic host cells. Suitable examples for selectable markers were described above and appropriate selection conditions for the individual selectable markers are also well-known in the prior art.

The expression vector may comprise more than one selectable marker gene and selection for the different selectable markers may be done simultaneously or sequentially for selecting host cells which are successfully transfected with the expression vector. The selection medium used for cultivation can comprise selection agents for all of the selectable markers of the expression vector. In another embodiment, cultivation can be performed first with a selection medium only comprising the selection agent(s) of one or a subset of the selectable marker genes of the vector, followed by the addition of one or more of the selection agents of the remaining selectable marker genes. In even another embodiment, the host cells are cultivated with a first selection medium only comprising the selection agent(s) of one or a subset of the selectable marker genes of the vector, followed by cultivation with a second selection medium comprising the selection agent(s) of one or more of the selection agents of the remaining selectable marker genes. According to one embodiment, the second selection medium does not comprise the selection agent(s) used in the first selection medium. One or more of the selectable marker genes of the expression vector preferably are amplifiable selectable marker genes. According to one embodiment in stage b), an antibiotic based selection step is followed by a selection step for an amplifiable marker gene such as DHFR.

The cultivation of stage b) preferably provides a plurality of eukaryotic host cells which are successfully transfected with the expression vector comprising a polynucleotide encoding the polypeptide of interest. Within this population of successfully transfected cells, i.e. cells that express the polypeptide of interest, the high expressing host cells must be identified. This is done in stages c) and d). If the cells were selected in stage b) e.g. as described above using a selection medium, the cells may be further cultivated in order to allow the cells to recover from selection prior to performing stage c).

### Stage C—Flow Cytometry Based Selection

In stage c), a first flow cytometry based selection step is performed. A plurality of eukaryotic host cells expressing the polypeptide of interest with desired yield is selected using flow cytometry. A selection step employing flow cytometry, in particular fluorescence activated cell sorting (FACS), has the advantage that large numbers of cells can be screened rapidly for the desired characteristic expression yield. Thus, as explained in the background of the present invention, flow cytometry based selection is suitable for screening a large number of cells for their expression characteristics. Thereby, the rare high expressing cell clones can be identified in the population of successfully transfected cells and separated from the low or no producing cells.

According to one embodiment, high expressing cells are identified by detecting the expression of a co-expressed reporter such as e.g. green fluorescence protein (GFP), CFP, YFP, luciferase or other common reporter that can be detected by flow cytometry. In such reporter based selection system, the expression of the reporter gene correlates with the expression of the polypeptide of interest. E.g. a reporter gene can be comprised on the same vector as the polynucleotide encoding the polypeptide of interest or can be comprised on a vector that is co-transfected with the vector comprising the polynucleotide encoding the polypeptide of interest. The reporter may be intracellularly located, thereby marking the expressing cell. According to one embodiment, the expression of the reporter is tightly linked to the expression of the polypeptide of interest. E.g. the reporter and the polypeptide of interest may be expressed as separate proteins but from the same expression cassette, however, separated by an IRES element (internal ribosomal entry site). Furthermore, the reporter and the polypeptide of interest may be expressed as fusion protein. According to one embodiment, in the expressing cassette for expressing the polypeptide of interest, the polynucleotide encoding the polypeptide of interest is separated by at least one stop codon from the polynucleotide encoding the reporter. A fusion protein comprising the reporter is only expressed if translation reads over the stop codon. As stop codon readthrough occurs only to a certain extent, which can be influenced by the number and design of the stop codon and the culture conditions (see also below), a certain proportion of the polypeptide of interest is produced as fusion protein comprising the reporter which can be detected by flow cytometry. The remaining proportion is expressed normally as polypeptide of interest. Using a respective strategy allows to tightly link the expression of the reporter to the expression of the polypeptide of interest. The more fusion protein is obtained, the higher is the expression of the polypeptide of interest. The principle of obtaining fusion proteins by stop codon read through will also be explained subsequently in conjunction with a preferred embodiment wherein a fusion protein is displayed on the cell surface and e.g. is stained by using a detection compound. For expressing a secreted polypeptide of interest it is preferred to additionally include a polynucleotide encoding a membrane anchor either between the stop codon and the polynucleotide encoding the reporter or downstream of the polynucleotide encoding the reporter. In case of secreted polypeptides of interest, the membrane anchor ensures that the reporter remains associated with the expressing cell either intracellularly or in the later case displayed on the cell surface when the fusion protein is expressed. Thereby the reporter comprised in the fusion protein provides the expressing cells with a trait that is selectable by flow cytometry. The polypeptide of interest is expressed into the culture medium. The higher e.g. the fluorescence, the more fusion protein is produced and accordingly, the higher is the expression rate of the polypeptide of interest. A respective method is e.g. disclosed in WO 03/014361 to which it is referred.

According to one embodiment stage c) comprises selecting a plurality of eukaryotic host cells expressing the polypeptide of interest with a desired yield based upon to the presence or amount of expressed polypeptide of interest using flow cytometry. Preferably, the polypeptide of interest is a secreted polypeptide. According to one embodiment, the polypeptide of interest is detected on the cell surface using a detection compound that binds to the polypeptide of interest. According to one embodiment, the secreted polypeptide of interest is detected while it passes the cell membrane and accordingly is transiently associated with the plasma membrane during polypeptide secretion. A respective flow cytometry based selection system is e.g. disclosed in WO 03/099996 to which it is referred. As the examples provided herein show, a respective FACS based selection system is suitable for use in conjunction with the present invention.

According to one embodiment, a portion of the polypeptide of interest is expressed fused to a membrane anchor and thus as membrane-bound fusion polypeptide. Thereby, a portion of the polypeptide is displayed as fusion polypeptide on the cell surface and can be stained using a detection compound. No reporter is required for this type of selection. Due to the presence of the membrane anchor, the polypeptide of interest is tightly anchored to the expressing cell which is an advantage over embodiments wherein the secreted polypeptide of interest is detected while it is transiently associated with the plasma membrane during polypeptide secretion. As the amount of produced fusion polypeptide correlates with the overall expression rate of the expressing cell, host cells can be selected via flow cytometry based upon the amount of fusion polypeptide displayed via the membrane anchor on the cell surface. This allows a rapid selection of high producing host cells. To allow efficient selection using flow cytometry, preferably by using FACS, it is advantageous to use special expression cassette designs for expressing the product of interest. Thus, according to one embodiment, the polynucleotide encoding the polypeptide of interest is comprised in an expression cassette that is designed such that a portion of the expressed polypeptide of interest comprises a membrane anchor. The polypeptide of interest which is fused to a membrane anchor is also referred to a fusion polypeptide or fusion protein. Several options exist to achieve that result.

According to one embodiment, the plurality of eukaryotic host cells provided in stage a) comprise a heterologous expression cassette comprising
i) the polynucleotide encoding the polypeptide of interest,
ii) at least one stop codon downstream of the polynucleotide encoding the polypeptide of interest, and
iii) a further polynucleotide downstream of the stop codon encoding a membrane anchor and/or a signal for a membrane anchor; and
   in stage b) cultivating the eukaryotic host cells is performed to allow expression of the polypeptide of interest wherein at least a portion of the polypeptide of interest is expressed as fusion polypeptide comprising a membrane anchor, wherein said fusion polypeptide is displayed on the surface of said host cell; and
   in stage c), the first, flow cytometry based selection comprises
selecting a plurality of eukaryotic host cells expressing the polypeptide of interest with a desired yield based upon the presence or amount of the fusion polypeptide displayed on the cell surface using flow cytometry.

Thus, according to this embodiment, the method comprises
a) providing a plurality of eukaryotic host cells comprising a heterologous expression cassette comprising
i) the polynucleotide encoding the polypeptide of interest,
ii) at least one stop codon downstream of the polynucleotide encoding the polypeptide of interest, and
iii) a further polynucleotide downstream of the stop codon encoding a membrane anchor and/or a signal for a membrane anchor;

b) cultivating the eukaryotic host cells to allow expression of the polypeptide of interest wherein at least a portion of the polypeptide of interest is expressed as fusion polypeptide comprising a membrane anchor, wherein said fusion polypeptide is displayed on the surface of said host cell;
c) performing a first, flow cytometry based selection stage, comprising
   selecting a plurality of eukaryotic host cells expressing the polypeptide of interest with a desired yield based upon the presence or amount of the fusion polypeptide displayed on the cell surface using flow cytometry;
d) performing a second, colony picking based selection stage, comprising
   obtaining single cell colonies in a medium which prevents the migration of the cells from a plurality of eukaryotic host cells selected in stage c);
   detecting within the obtained cell colonies one or more cell colonies having desired colony characteristics;
   picking one or more cell colonies having the desired colony characteristics; and
e) cultivating the picked cell colonies to provide cell clones.

Transcription of the polynucleotide comprised in the expression cassette results in a transcript comprising in consecutive order at least
a polynucleotide, wherein translation of said polynucleotide results in the polypeptide of interest;
at least one stop codon downstream of said polynucleotide;
a polynucleotide downstream of said stop codon, encoding a membrane anchor and/or a signal for a membrane anchor.

At least a portion of the transcript is translated into a fusion polypeptide comprising the polypeptide of interest and the membrane anchor by translational read-through of the at least one stop codon. This design of the expression cassette that is used in this embodiment has the effect that through translational read-through processes (the stop codon is “leaky”) a defined portion of the polypeptide of interest is produced as a fusion polypeptide comprising a membrane anchor. Thus, at least a portion of the transcript is translated into a fusion polypeptide comprising the polypeptide of interest and the membrane anchor by translational read-through of the at least one stop codon. Translational read-through may occur naturally due to the choice of the stop codon/design of the translation termination signal or can be induced by adapting the culturing conditions, e.g. by using a termination suppression agent. As a result, the fusion polypeptide is being displayed on the cell surface and cells displaying high levels of membrane-anchored fusion polypeptide can be selected by flow cytometry, preferably by FACS. Thereby, host cells are selected that express the polypeptide of interest with high yield. Details and preferred embodiments of this stop codon readthrough based technology are described in WO 2005/073375 and WO 2010/022961 and it is referred to this disclosure for details. The expression cassette may comprise only a single stop codon upstream of the coding sequence for the membrane anchor (or signal for a membrane anchor). However, it is also possible to use a series of two or more stop codons, e. g. two or three, or four stop codons, which may be the same or different. Also the context of the stop codon, i.e. the trinucleotide stop codon itself as well as the nucleotide(s) respectively codon immediately downstream of the stop codon, has an influence on the read-through levels. However, it needs to be ensured that a certain level of translational read-through still occurs in order to allow the production of the fusion polypeptide which may be achieved according to one embodiment by adjusting the culture conditions. Suitable translation termination signals and thus stop codons and stop codon settings with incomplete translation termination efficiency can be designed as described in the prior art (see e.g. Li et al., 1993, Journal of Virology 67 (8), 5062-5067; McCughan et al., 1995, Proc. Natl. Acad. Sci. 92, 5431-5435; Brown et al., 1990, Nucleic Acids Research 18 (21), 6339-6345, herein incorporated by reference). The additional amino acids that are incorporated into the fusion polypeptide due to the read-through of the stop codon can be of any kind as long as the fusion protein is displayed on the cell surface. As said additional amino acids are only incorporated into the fusion polypeptide, the amino acid sequence of the polypeptide of interest remains unaltered. In addition to the possible use of multiple stop codons following the polynucleotide encoding the polypeptide of interest, it will normally be advantageous to use multiple stop codons downstream of the sequence encoding the membrane anchor or signal for membrane anchor. The use of multiple stop codons in this position, e. g. up to about ten stop codons, such up to about six or eight stop codons, such as about two, three, four or five stop codons, will ensure efficient termination of translation. The primary transcript may be a pre-mRNA comprising introns. A respective pre-mRNA would be processed (spliced) into mRNA. Alternatively, transcription may result directly in mRNA if no introns are present. During translation of the mRNA transcript there is usually a natural level of background read-through of the stop codon(s) or a respective read-through level can be induced by adapting the culture conditions. This read-through level results in a certain proportion of fusion polypeptides. These fusion polypeptides comprise a membrane anchor, which tightly anchors the fusion polypeptides to the cell surface.

According to one embodiment, the expression cassette comprises iv) a polynucleotide encoding a reporter, such as e.g. GFP. Said polynucleotide encoding the reporter is located downstream of the stop codon. Upon stop codon read-through a fusion polypeptide is obtained which comprises the reporter, thereby allowing selection by flow cytometry based on the characteristics of the reporter such as e.g. its fluorescence. Details of said embodiment were already described above and it is referred to the above disclosure. Preferably, the polynucleotide encoding the reporter is located downstream of the polynucleotide encoding a membrane anchor and/or a signal for a membrane anchor.

According to an alternative embodiment, the plurality of eukaryotic host cells provided in stage a) comprise a heterologous expression cassette comprising
i) the polynucleotide encoding the polypeptide of interest,
ii) an intron comprising a 5′ splice donor site and a 3′ splice acceptor site and comprising an in frame translational stop codon and a polyadenylation signal and
iii) a polynucleotide downstream of said intron encoding a membrane anchor and/or a signal for a membrane anchor; and
   in stage b) cultivating the eukaryotic host cells is performed to allow expression of the polypeptide of interest wherein at least a portion of the polypeptide of interest is expressed as fusion polypeptide comprising a membrane anchor, wherein said fusion polypeptide is displayed on the surface of said host cell; and
   in stage c) the first, flow cytometry based selection comprises
selecting a plurality of eukaryotic host cells expressing the polypeptide of interest with a desired yield based upon the presence or amount of the fusion polypeptide displayed on the cell surface using flow cytometry.

Thus, according to this embodiment, the method comprises
a) providing a plurality of eukaryotic host cells comprising a heterologous expression cassette comprising
i) the polynucleotide encoding the polypeptide of interest,
ii) an intron comprising a 5′ splice donor site and a 3′ splice acceptor site and comprising an in frame translational stop codon and a polyadenylation signal and iii) a polynucleotide downstream of said intron encoding a membrane anchor and/or a signal for a membrane anchor;
b) cultivating the eukaryotic host cells to allow expression of the polypeptide of interest wherein at least a portion of the polypeptide of interest is expressed as fusion polypeptide comprising a membrane anchor, wherein said fusion polypeptide is displayed on the surface of said host cell;
c) performing a first, flow cytometry based selection stage, comprising
   selecting a plurality of eukaryotic host cells expressing the polypeptide of interest with a desired yield based upon the presence or amount of the fusion polypeptide displayed on the cell surface using flow cytometry;
d) performing a second, colony picking based selection stage, comprising
   obtaining single cell colonies in a medium which prevents the migration of the cells from a plurality of eukaryotic host cells selected in stage c);
   detecting within the obtained cell colonies one or more cell colonies having desired colony characteristics;
   picking one or more cell colonies having the desired colony characteristics; and
e) cultivating the picked cell colonies to provide cell clones.

This design of the expression cassette that is used in this embodiment has the effect that through transcription and transcript processing at least two different mature mRNAs (mRNA-polypeptide of interest) and (mRNA-polypeptide of interest-anchor) are obtained from the expression cassette. Translation of the mRNA-polypeptide of interest results in the product of interest. Translation of the mRNA-polypeptide of interest-anchor results in a fusion polypeptide comprising the product of interest and a membrane anchor. As a result, this fusion polypeptide is again displayed on the cell surface and cells displaying high levels of membrane-anchored fusion polypeptide can be selected by flow cytometry, preferably FACS. Thereby, again host cells can be selected that have a high expression rate. Details and embodiments of this intron based technology are described in WO 2007/131774. It is referred to this disclosure. According to one embodiment, the expression cassette comprises iv) a polynucleotide encoding a reporter, such as e.g. GFP. Said polynucleotide encoding the reporter is located downstream of the intron. Thereby, a fusion polypeptide is obtained which comprises the reporter, thereby allowing selection by flow cytometry based on the characteristics of the reporter such as e.g. its fluorescence. Preferably, the polynucleotide encoding the reporter is located downstream of the polynucleotide encoding a membrane anchor and/or a signal for a membrane anchor. Thereby, the reporter is located inside the host cell.

Both exemplary embodiments described above result in that a portion of the polypeptide of interest is expressed as fusion polypeptide that is displayed at the surface of the host cells, and cells displaying high levels of membrane-anchored fusion polypeptides (indicating a high level of secreted polypeptide) can be selected e.g. by flow cytometry, in particular by fluorescence activated cell sorting (FACS). Here, different embodiments are available. E.g. if a reporter is comprised in the fusion polypeptide, high expressing host cells can be selected based upon a characteristic of the reporter, e.g. its fluorescence.

According to one embodiment, cells are contacted with an appropriately labelled detection compound that binds the fusion protein, e.g. the portion corresponding to the polypeptide of interest.

The amount of fusion polypeptide present and thus detectable on the cell surface usually increases during polypeptide synthesis as the fusion polypeptide remains anchored to the cell membrane and thus accumulates on the cell surface as expression continues. According to one embodiment, the expression cassette is constructed such that approximately ≦50%, ≦25%, ≦15%, ≦10%, ≦5%, ≦2.5%, ≦1.5%, ≦1% or less than ≦0.5% fusion polypeptide is obtained. The remaining portion is produced as the secreted polypeptide form not comprising the membrane anchor. As described, when using an expression cassette that obtains the fusion polypeptide based on stop codon read through, the level of stop codon read through can be influenced by the choice and number of the stop codon(s) and the regions adjacent to the stop codon, in particular the nucleotide following the stop codon, as well as by the culture conditions used during stage c). When using a splicing based system, the amount of fusion polypeptide can be controlled by the design and size of the intron. The general advantage of a rather low amount of obtained fusion polypeptide is a higher stringency in the subsequent selection/enrichment and flow sorting procedure, which is preferably done by FACS, leading to a better resolution of high producing versus ultra high producing clones. If too much polypeptide is displayed, saturation of the cell surface capacity for membrane bound polypeptides might occur, which may render discrimination of expression levels, in particular of high expression levels more difficult. Therefore, a rather low expression of the fusion polypeptide is advantageous in order to select ultra high expressing clones in stage c). Accordingly, preferably only ≦15%, ≦10%, ≦5%, ≦2% or even ≦1.5% of the polypeptide of interest is produced as fusion polypeptide. However, when using a leaky stop codon based selection system in stage c) it is also possible to conditionally increase the read-through level if necessary/desired, e.g. by using a termination suppression agent during culturing. The use of a termination suppression agent in the culture media during stage c) is one way of influencing the level of stop codon read through by the culture conditions. A termination suppression agent is a chemical agent which is able to suppress translational termination resulting from the presence of a stop codon. In particular, the termination suppression agent is an antibiotic belonging to the aminoglycoside group. Aminoglycoside antibiotics are known for their ability to allow insertion of alternative amino acids at the site of a stop codon, thereby resulting in “read-through” of a stop codon or stop codon setting that otherwise normally would result in translation termination. Aminoglycoside antibiotics include G-418, gentamycin, paromomycin, hygromycin, amikacin, kanamycin, neomycin, netilmicin, streptomycin and tobramycin. However, as a low read-through level is advantageous, flow cytometry based selection in stage c) is preferably performed in the absence of a termination suppression agent.

The membrane anchor may be of any kind as long as it enables anchorage of the polypeptide of interest to the cell membrane and thus allows the display of the fusion polypeptide on the cell surface. Suitable embodiments include but are not limited to a GPI anchor or a transmembrane anchor. A transmembrane anchor is preferred to ensure tight binding of the fusion polypeptide to the cell surface and to avoid shedding. Particularly preferred, in particular when expressing antibodies as polypeptide of interest, is the use of an immunoglobulin transmembrane anchor. Other suitable membrane anchors and preferred embodiments of an immunoglobulin transmembrane anchor are described in WO 2007/131774, WO 2005/073375 and WO 2010/022961.

According to one embodiment, selection stage c) comprises contacting the host cells with a detection compound binding the displayed fusion polypeptide and selecting a plurality of host cells based upon the presence or amount of the detection compound bound to the cell surface. The detection compound used for binding to the fusion polypeptide may have at least one of the following characteristics:
said compound is labelled;
said compound is fluorescently labelled;
said compound is an antigen;
said compound is an immunoglobulin molecule or a binding fragment thereof;
said compound is protein-A, -G, and/or -L.

The detection compound used for binding the fusion polypeptide at the cell surface can for example be an immunoglobulin molecule or a fragment thereof such as an antibody or antibody fragment, recognising the fusion polypeptide. Basically all accessible portions of the fusion polypeptide can be detected, thereunder also the portion corresponding to the polypeptide of interest which is secreted in parallel to the fusion polypeptide in soluble form. According to one embodiment, the detection compound is an antigen. This embodiment is suitable, if the expressed polypeptide of interest is for example immunoglobulin molecule or a fragment thereof such as an antibody, binding the respective antigen.

In order to allow detection and selection, said detection compound used for binding the fusion polypeptide may be labelled. The labelled detection compound that binds the fusion polypeptide displayed on the cell surface thereby labels respectively stains the cell surface. The higher the amount of fusion polypeptide that is expressed by the host cell, the more labelled detection compound is bound. This has the advantage that the flow cytometry based selection of the host cells can be easily performed as not only the presence but also the amount of the bound detection compound can be determined due to the label. To select high producing host cells, those host cells are selected from the population of host cell which are most effectively respectively intensively labelled by the detection compound. The label must by suitable for flow cytometry based selection, in particular FACS selection. A fluorescent label is preferred as this allows easy detection by flow cytometry. Suitable fluorescent labels are known to the skilled person.

While one flow cytometry based selection cycle is sufficient, according to one embodiment, two or more selection cycles according to stage c) may be performed to select high expressing eukaryotic host cells. According to one embodiment, selection is based on the degree of binding of the detection compound to the cell surface. Thus, eukaryotic host cells may be selected in each selection cycle based upon the amount of bound detection compound. According to another embodiment, selection is based upon the degree of expression of the reporter if used (see above), e.g. is based on its fluorescence.

Accordingly, the flow cytometry selected cells can be subjected to a second round or further rounds of flow cytometry based selection. Furthermore, a threshold can be set for defining which host cells shall be selected and preferably sorted by FACS. Thus, those host cells that were most effectively/intensively labelled can be selected based upon the degree respectively amount of cell surface staining. E.g. the top 15%, top 10%, top 5% or the top 2% of the host cells can be selected in stage c) for further use in stage d).

Preferably, a population of high producing cells is enriched in flow cytometry based selection stage c), preferably based on the degree of binding of the detection compound to the cell surface, in particular bound to the fusion polypeptide.

In a preferred embodiment, host cells expression a high amount of polypeptide of interest which accordingly depict a high signal are sorted using fluorescence-activated cell sorting (FACS). In the context of the present invention, FACS sorting is particularly advantageous, since it allows rapid screening of large numbers of host cells to identify and enrich those cells which express the polypeptide of interest with a high yield. This embodiment is particularly suitable if the cells are selected based upon the expression of a fusion protein as described above. As according to the preferred embodiment approximately only 10% or less or only 5% or less of the polypeptide is produced as a fusion polypeptide, a higher fluorescence detected would correspond to a higher expression also of the polypeptide of interest, which can be e.g. secreted into the culture medium. Those cells, showing the highest fluorescence rate can be identified and isolated by FACS. A positive and statistically significant correlation between fluorescence, as determined by FACS, and the amount of produced polypeptide is found. Therefore, FACS sorting can be used not only for a qualitative analysis to identify cells expressing a polypeptide of interest in general, but can actually be used quantitatively to identify those host cells that express high levels of the polypeptide of interest. Therefore, high-producing host cells can be selected/enriched e.g. based on the degree of binding of the labelled detection compound to the fusion polypeptide, which is anchored to the cell surface. Thereby the best producing cells can be selected/enriched in stage c). This leads to a significant reduction of non-producing clones in the selected cell populations.

It is preferred to select cells which express the polypeptide of interest with the desired yield as pool. E.g. several high expressing cells, e.g. at least 10, at least 20, at least 30, at least 50, at least 100, at least 200, at least 300, at least 500, at least 1000 or at least 5000 high expressing cells can be selected in stage c) and sorted into a cell pool. This cell pool comprising a plurality of different high expressing cells is also referred to as high expressing cell pool. This embodiment is particularly advantageous as said cell pool comprising different individual cells can then be used in stage d) to obtain individual cell colonies.

As explained above, a flow cytometry based selection has the advantage that a high number of cells can be screened within short time. Based on this high throughput selection, a population or pre-selected cells is provided which express the polypeptide of interest with high yield. Said population of pre-selected high producers is then subjected to stage d).

### Stage D—Colony Picking Based Selection

In stage d), eukaryotic host cells that were selected in stage c) are used for obtaining single cell colonies in a medium which prevents migration of dividing cells. Therefore, single cell colonies are obtained from eukaryotic cells selected in stage c). Thus, stage d) advantageously starts from cells that express the polypeptide of interest with high yield because such cells were selected in stage c). For this purpose, a solid or semi-solid medium can be used. Within the obtained cell colonies, one or more cell colonies having desired colony characteristics are detected. The one or more cell colonies having the desired colony characteristics are then picked and e.g. transferred into a new reaction vessel, e.g. into a well of a multi-well plate. Preferably, a plurality of cell colonies is picked in stage d) in order to increase the clone diversity. As described herein, the picked colonies are then cultivated in stage e) and e.g. analysed regarding their characteristics.

For performing stage d), standard clone picking systems such as e.g. the ClonePix robotic systems (GENETIX/Molecular Device) can be used. Non-limiting suitable embodiments will be described in the following.

In stage d), single cell colonies are obtained from eukaryotic host cells that were selected in stage c). Stage d) can be performed with the entire eukaryotic host cells selected in stage c) or with only a part of the eukaryotic host cells selected in stage c). For example, up to about 5%, up to about 10%, up to about 20%, up to about 30%, up to about 40%, up to about 50%, up to about 60%, up to about 70%, up to about 80%, up to about 90%, or up to about 100% of the eukaryotic host cells selected in stage c) may be used in stage d). The part of the eukaryotic host cells selected in stage c) which are used in stage d) may be chosen arbitrarily or by a further selection step. The eukaryotic host cells obtained in stage c) may hence be selected for further characteristics before using them in stage d). In preferred embodiments, however, no further selection step is performed between stage c) and stage d).

In certain embodiments, the plurality of eukaryotic host cells selected in stage c) or a part thereof may be cultivated before using them in the selection stage d). Such cultivation step may allow recovery from FACS selection. The term “eukaryotic host cells selected in stage c)” also encompasses offspring or descendants of the eukaryotic host cells directly obtained in stage c), as well as cells derived therefrom. According to one embodiment, no intermediate cultivation step is performed between stage c) and d).

The single cell colonies which are to be obtained in stage d) are cell colonies derived from one single ancestral cell. Single cell colonies are clonal colonies wherein all cells in the colony are clones of each other. Preferably, all the cells in a single cell colony contain the same or substantially the same genetic information. The single cell colonies are obtained in stage d) by singling out the plurality of eukaryotic host cells selected in stage c) and cultivating and/or proliferating them in a medium which prevents the migration of the cells. In preferred embodiments, the plurality of eukaryotic host cells selected in stage c) is added to the medium which prevents the migration of the cells in a concentration that enables the growth of single cell colonies. In particular, the concentration is such that two single cell colonies grown from two eukaryotic host cells selected in stage c) do not touch each other in the medium which prevents the migration of the cells. For example, less than 20%, preferably less than 15%, less than 10%, less than 5% or less than 2% of the single cell colonies obtained in stage d) touch another cell colony. Preferably, on average two single cell colonies in the medium which prevents the migration of the cells are separated from each other by at least one colony diameter, preferably at least two, at least three, at least four, at least five, at least seven, at least 10, at least 15, at least 20, or at least 25 colony diameters. The concentration of the plurality of eukaryotic host cells selected in stage c) may be adjusted before adding it to the medium which prevents the migration of the cells, e. g. by dilution or enrichment. In certain embodiments, the medium which prevents the migration of the cells is seeded with the plurality of eukaryotic host cells selected in stage c) at a concentration in the range of about 10 to about 1000 cells/ml, preferably about 25 to about 500 cells/ml, more preferably about 100 to about 300 cells/ml, most preferably about 150 to about 250 cell/ml, in particular about 200 cells/ml.

The medium which prevents the migration of the cells in particular is a semi-solid medium or a solid medium. Suitable solid or semi-solid media are known in the art. Preferably the medium is a methylcellulose medium which comprises, for example, 0.1% to 10% methylcellulose, preferably 1% to 4%, more preferably 2% to 3% methylcellulose. Methylcellulose media may, for example, be obtained from Sigma-Aldrich Company Ltd. (Dorset, UK) under catalogue number M0387 (methylcellulose viscosity 1,500 cP (2% aqueous solution, 20° C.), CAS Number 9004-67-5) or catalogue number M0512 (methylcellulose viscosity 4,000 cP (2% aqueous solution, 20° C.), CAS Number 9004-67-5). However, also other solid or semi-solid media such as agar or agarose media may be used. The medium may further comprise growth factors and/or other supplements optimised to support the selection, survival and growth of the host cells. Suitable embodiments are well-known in the art.

For obtaining single cell colonies, the plurality of eukaryotic host cells selected in stage c) may be applied on top of or into the medium which prevents the migration of the cells. If a semi-solid medium is used, the host cells are preferably added into the medium. In case of a solid medium, the host cells are preferably placed on the surface of the medium. The host cells in/on the medium are then cultivated and/or proliferated for colony growth. In particular, the host cells are incubated under conditions and for a time interval suitable for colony growth of the specific eukaryotic host cells used. Potentially important conditions are, for example, temperature, humidity, CO₂ and O₂ concentration. Suitable conditions and incubation times are known in the art and are in particular dependent on the host cells used.

After growth of the cell colonies, one or more colonies having desired colony characteristics are detected. The colony characteristics that are used for selecting and thus picking cell colonies in stage d) include but are not limited to characteristics of the expression of the polypeptide of interest such as the yield of the polypeptide and/or the secretion of the polypeptide of interest, growth characteristics as well as further characteristics of the cell colony. According to one embodiment the selection criteria for picking include the colony size, the colony shape and/or the size and shape of the cells in the colony. Preferably, suitable cell colonies are at least selected based on their expression characteristics, in particular the yield of expressed polypeptide of interest and/or their growth characteristics. Preferably, the colony size and/or shape is considered to identify and thus pick cell colonies which have favourable characteristics.

For determining the expression characteristics, preferably a detection compound capable of associating with and in particular binding to the polypeptide of interest is used. The detection compound may for example be a natural binding partner of the polypeptide of interest or an antibody directed against the polypeptide of interest. Examples of the detection compound include but are not limited to antibodies, antibody fragments, peptides, lectins, receptors and other binding proteins. In case the polypeptide of interest is an antibody, the detection compound may be an antigen of said antibody or it may be a binding agent which binds said antibody. The detection compound itself may provide a detectable signal or may be capable of associating with, in particular binding to a compound providing the detectable signal. Suitable detectable signals are, for example, fluorescence signals, luminescence signals, colorimetric signals, phosphorescence signals and radioactive signals. In preferred embodiments, the detection compound comprises a fluorophore or is capable of associating with or binding to a further compound comprising a fluorophore. Furthermore, the detectable signal may be provided by a chemical reaction catalysed by the detection compound or a further compound which is capable of associating with, in particular binding to the detection compound. Particular examples are enzymatic reactions such as peroxidase reactions which result in the generation of a luminescent or fluorescent signal. In certain embodiments, the detectable signal is only generated or is changed upon binding of the detection compound to the polypeptide of interest. In further embodiments, detection compounds and/or compounds providing the detectable signal which are not associated with or bound to the polypeptide of interest are removed prior to measuring the detectable signal, for example by a washing step. In even further embodiments, the detection compounds and/or the compounds providing the detectable signal passively defuse through the medium and accumulate where they associate with or bind to the polypeptide of interest. The concentration of the detection compound and/or the intensity of the detectable signal increases at the sites of accumulation and thereby, the presence and amount or concentration of the polypeptide of interest can be located and determined. Furthermore, detection agents described above in conjunction with stage c) can be used in stage d). According to one embodiment, the same detection agent is used in stage d) as was used in stage c).

In further embodiments, the detectable signal is provided by a reporter. Suitable examples of reporters and embodiments were described above in conjunction with stage c). E.g. as described above, a portion of the polypeptide of interest can be expressed as fusion polypeptide comprising the reporter or the reporter can be expressed separately from the polypeptide of interest and remain intracellularly, thereby marking the cell. The detectable characteristic of the reporter such as e.g. its fluorescence can be used in stage d) to identify cell colonies that express the fusion polypeptide which comprises the reporter and thus expresses the polypeptide of interest with high yield. The higher or stronger the reporter characteristic, e. g. the fluorescence, the higher is the expression rate of the reporter which correlates with the expression of the polypeptide of interest.

Furthermore, the strategies described above in stage c) that allow the identification of cells that express the polypeptide of interest with high yield using flow cytometry can in essence also be used to identify high expressing cell colonies in the colony picking stage d). E.g. cells expressing a portion of the polypeptide of interest as membrane-anchored fusion polypeptide that is displayed on the cell surface can be stained using an appropriate detection compound and high expressing cell colonies can then be identified and picked based on the amount of bound detection compound. It is referred to the above disclosure for details.

The detectable signal is then associated with the respective cell colony. The colony characteristics, in particular the expression yield are then derived from the detected signal, in particular from the intensity and/or location of the detectable signal. The intensity of the detectable signal in particular correlates with the yield of the polypeptide of interest. Hence, an intensity of the detectable signal which is above a certain threshold can be indicative for a desired high yield of the polypeptide of interest. In certain embodiments, the intensity of the detectable signal in specific areas, in particular at or in the surrounding of the cell colony is considered.

In embodiments wherein a portion of the polypeptide of interest comprises a membrane anchor and remains bound to the cell membrane after expression whereas the remaining is expressed as secreted polypeptide of interest, the detectable signal preferably is measured at the cell colony. Preferably, the technology and features for expressing at least a portion of the polypeptide of interest as a fusion polypeptide comprising a membrane anchor as described herein can be used in this embodiment. Thereby, the yield of the secreted polypeptide of interest can be determined based on the portion of the polypeptide of interest expressed as a fusion polypeptide comprising a membrane anchor.

In embodiments where the polypeptide of interest is secreted into the medium and no fusion protein is produced, the detectable signal may be measured in the area surrounding the cell colony, and optionally additionally in the area of the cell colony. In specific embodiments, secreted polypeptides of interest form a halo or aura around the cell colony expressing them. This is in particular the case if the movement of the polypeptide of interest in the medium is reduced by a binding agent capable of binding to the polypeptide of interest. In general, with such a halo or aura, the polypeptide of interest is present in an area or zone which is associated with, coincident with, or around the cell or colony. Preferably, the aura or halo extends beyond the boundaries of the cell or colony. Preferably, the aura or halo as defined by the presence of the polypeptide of interest extends to 1, 2, 3, 4, 5, 10, 20 or 30 or more colony diameters beyond the boundaries of the colony. The yield of the polypeptide of interest is then only determined by the detectable signal measured in the respective area. Furthermore, in order to determine whether a desired level of secretion of the polypeptide of interest is reached, a ratio or difference between the intensity of the detectable signal in the surrounding area of the cell colony and at the cell colony can be calculated. Respective principles are well-known in the prior art for cell colony picking systems.

In certain embodiments where the polypeptide of interest is secreted by the host cells and wherein preferably no fusion polypeptide is obtained, the movement of the polypeptide of interest in the medium is reduced by a binding agent capable of binding to the polypeptide of interest. In particular, the binding agent forms a complex with the polypeptide of interest, thereby increasing the overall size of the polypeptide of interest and thus, decreasing its mobility in the solid or semi-solid medium. In particular embodiments, the binding agent forms oligomeric or multimeric complexes with the polypeptide of interest, preferably comprising two or more, three or more, four or more or five or more polypeptides of interest per complex. In the further embodiments, the polypeptide of interest is precipitated when binding to the binding agent. Using such a binding agent, a closer association between the secreted polypeptide of interest and the cell colony expressing it can be obtained. Thereby, the detectable signal of a polypeptide of interest can more easily be assigned to the cell colony which expresses the polypeptide of interest. In certain embodiments, the formation of a polypeptide/binding agent complex in the form of an aura or halo increases the effective concentration of the polypeptide of interest in the particular area. Such an increased effective concentration enables more efficient binding by the detection compound to the polypeptide of interest. Therefore, the amount of detection compound which is required is less than in the absence of the use of a binding agent. Examples of the binding agent include but are not limited to antibodies, antibody fragments, peptides, lectins, receptors and other binding proteins. These binding proteins may optionally be modified to increase their size and/or to decrease their mobility in the medium.

In certain embodiments, the detectable signal is compared to a threshold level and an intensity of the detectable signal equal to our above the threshold level indicates a desired yield and/or a desired secretion level of the polypeptide of interest. According to one embodiment, the detectable signal is only measured in the area as defined above, e.g. only at or in the area of the cell colony or only in the area surrounding the cell colony. The detectable signal may be adjusted by the background signal prior to comparison with the threshold level. The threshold level may be a predetermined threshold level or it may be calculated or derived from the intensities of the detectable signal of all or a subset of the obtained cell colonies.

By analysing the detectable signal, in particular the intensity and/or location of the detectable signal, the expression characteristics of the polypeptide of interest for the cell colony associated with the detectable signal can be determined.

In preferred embodiments, further characteristics of the cell colony are determined and considered for colony picking. In particular, the size of the colony can be determined and used as basis for cell colony selection. The size of the colony in particular refers to its diameter or to the number of cells in the colony. A larger size of the colony indicates a higher proliferation rate of the cell clone forming the colony, thereby indicating favourable growth characteristics. Since a higher proliferation rate of the cell clone reduces the cultivation time during the final production process of the polypeptide of interest, a higher proliferation rate generally is desired. Hence, the colony size preferably is compared to a threshold level and colony sizes above said threshold level are indicative for a desired proliferation rate of the cell clone. The threshold level may be a predetermined threshold level or it may be calculated or derived from the sizes of all or a subset of the obtained cell colonies. Furthermore, also the shape of the cell colony may be detected. A symmetrical, round shape of the cell colony is indicative for a single cell colony which originates from one single cell. Such single cell colonies can be distinguished from cell colonies originating from two or more cells by the colony shape, because cell colonies originating from two or more different cells have an irregular or asymmetrical shape. Colony size and shape are preferably detected by taking an image of the colony. Preferably, white light is used for imaging the cell colonies. Furthermore, the cells may be stained with a suitable marker and the signal of said cell marker may be detected.

In further embodiments, specific markers and/or polypeptides other than the polypeptide of interest expressed by the cells of the cell colonies may be determined, in particular by specific binding agents comprising or capable of binding to a signalling agent. Thereby, the type of the cells, specific expression patterns of the cells and/or the expression of undesired polypeptides, for example polypeptides which potentially contaminate the polypeptide of interest, can be determined.

According to one embodiment, the colony picking based selection in stage d) is not based on expression criteria. According to one embodiment, colony picking based selection in stage d) is based only on growth characteristics of the cell colonies.

By analysing the expression characteristics of the polypeptide of interest and/or characteristics of the cell colony such as growth characteristics, cell colonies can be detected and selected which have the desired colony characteristics. The type, number and combination of desired colony characteristics which are considered when detecting and selecting the cell colonies can be set as suitable for the colony picking based selection. For example, cell colonies can be detected based upon a desired expression of the polypeptide of interest, growth characteristics, a desired colony size and/or a desired colony shape. In these embodiments, a threshold value may be set for each characteristic as appropriate, so that every colony considered for picking may be above or below each threshold as desired. In certain embodiments, the cell colonies are detected and selected based at least upon a desired yield of the polypeptide of interest and optionally on a desired colony size and optionally additionally on a desired colony shape.

In preferred embodiments, any of the steps set out in relation to the colony picking based selection, such as contacting the polypeptide of interest with a detection compound, measuring the detectable signal, imaging the colonies, as well as associated steps such as selection and/or picking of cells or colonies of interest may be conducted using automated robotic apparatuses. In certain embodiments, the robotic apparatus comprises a ClonePix FL apparatus (Genetix, New Milton, United Kingdom). Features of a robotic apparatus which are advantageous for the performance of the methods described here, and which are present in the ClonePixFL apparatus, include any one or more of the following: cool white light illumination; up to 5 fluorescence combinations; high-resolution cooled CCD camera; ability to image at standard pixel resolution of 7 μm permitting fluorescent detection of colonies with as few as 10 cells; image zooming to 1 μm resolution for detailed colony inspection; ability to pick colonies at up to 400 clones per hour; easy-to-use custom software (ExCellerate) for intelligent picking, Halo Recognition, barcoding and clone-by-clone data tracking; stackers hold up to 10 source and collection plates, and optional Class II-type containment. Respective clone picking apparatuses are for example described in EP 1 752 771 and EP 1 754 537. An apparatus for picking cell colonies in particular may comprise an apparatus bed for arranging a sample container comprising a plurality of cell colonies; a camera for capturing images of the cell colonies; an image processor for identifying cell colony locations from captured images; and a picking head movable around the apparatus bed using positioning motors to cell colony locations identified by the image processor, wherein the picking head comprises one or more, in particular a plurality of hollow pins connected through fluid conduits to a pressure controller that is operable to aspirate quantities of medium from the sample container into the hollow pins, to retain the medium and to expel it when required, thereby allowing cell colonies to be picked from the medium. The apparatus preferably further comprises a fluorescence detection system.

One or more of the cell colonies having the desired colony characteristics are then picked in stage d). Picking in this respect preferably means that all or a part of the cells of a colony having the desired colony characteristics are transferred into a container such as e.g. a reaction tube, a cell culture dish, a cell culture flask or a well of a multi-well plate. Each selected single cell colony is transferred into a separate container. The transfer may be achieved e. g. by aspirating the cells into a hollow pin and dispensing the aspirated cells from the hollow pin into the container.

Since single cell colonies are obtained by the colony picking based selection of stage d), all cells in a single cell colony are genetically identical. Hence, each picked cell colony represents a cell clone.

### Stage E—Cultivation of the Picked Colonies

After picking of the cell colonies selected in stage d), they are cultivated to provide cell clones, in particular in the form of clonal cell cultures. A clonal cell culture is a cell culture derived from one single ancestral cell. In a clonal cell culture, all cells are clones of each other. Preferably, all the cells in a cell culture contain the same or substantially the same genetic information. The cell colonies picked in stage d) are cultivated for cell growth. In particular, the cells are incubated in a cell culture medium under conditions and for a time interval suitable for cell growth of the specific eukaryotic host cells used. The cell culture medium may be a fluid, semi-solid or solid medium and preferably is a fluid medium. According to one embodiment, cultivation occurs in the absence of selection pressure. According to a preferred embodiment, a selection medium is used for cultivation which allows to maintain the selection pressure for at least one of the selectable markers that were initially used for selection. Suitable cell culture media, conditions and incubation times are also known in the art. Cultivation may be performed, for example, in wells of a multi-well plate or in culture dishes or culture flasks. Preferably, a multi-well plate is used.

The picked colonies are preferably cultivated in an incubator. Preferably, the picked colonies are cultivated in an incubator of an automated cell handling system such as a cloning robot. Preferably, the automated cell handling system is adapted to receive multi-well plates, in particular 96-well plates.

According to one embodiment, stage e) comprises selecting cell clones for their productivity performance.

In certain embodiments of stage e), the amount of cells is monitored during the cultivation and/or is determined after specific cultivation times. According to one embodiment, the cell growth of the picked colonies that are cultivated in stage e) is determined. The number of cells in the culture can be determined by any known method, for example by cell counting in a defined sample of the cell culture and/or by measuring the optical density of the cell culture and/or by imagining the cell culture, e.g. using a cell confluence imager. Thereby, the proliferation rate of the cells of the picked colonies can be analysed. Furthermore, the cell viability may be determined using commonly known methods such as staining of apoptotic or dead cells in a sample of the cell culture.

In certain embodiments, the amount or concentration of the polypeptide of interest in the cell culture is determined, in particular at a specific cultivation time or a specific cell density. The amount or concentration of the polypeptide of interest is preferably determined in a sample of the cell culture using detection methods known in the art. According to one embodiment, a sample of the obtained cell culture is removed for determining the amount of expressed polypeptide of interest using methods known in the prior art such as HPLC. E.g. the titer can be measured by analysing the culture supernatant. E.g. when the cells achieve approx. the same cell density in culture, the supernatants are collected and the protein amount in the supernatant is determined. The protein expressed in the supernatant essentially corresponds to the protein of interest.

According to one embodiment, in the automated cell handling system the clones are controlled for growth by a cell confluence imager and are then diluted by a liquid handling system of the system.

According to one embodiment, one or more daughter cultures are prepared from the parent culture that is obtained from cultivating the picked colonies. Said daughter cultures can be used e.g. for analysing the expression yield and/or the growth characteristics or can be stored as back-up.

Based on the expression rate of the polypeptide of interest and optionally the proliferation rate of the host cells, the productivity performance of the cultivated cell clone can be determined. Cell cultivation and determination of the expression rate and proliferation rate preferably are performed by an automated or semi-automated process, e.g. using a cloning robot.

According to one embodiment, after determining the productivity performance of each individual clone, a titer ranking is made to select the best producing clones. In preferred embodiments, cell clones having a high productivity performance are selected for large scale production processes of the polypeptide of interest.

As described above, the combination with the incubation in an automated cell handling system is particularly preferred. Thereby, a unique combination of three top in class technologies is integrated in the cell line development platform to achieve a fast, selective, efficient and high throughput cell cloning system. The flow cytometry sorting (FACS) allows a very stringent high throughput selection of high producing cells for example by way of a specific fluorescence labelling of membrane bound expressed polypeptides. The clone picking step that is performed with the respectively preselected high producing cells ensures the accurate selection of the most effected secretors which have good growth characteristics. The subsequently used automated cell handling system is supporting the parallel handing of a large amount of high producing clones identified by combining the selections described in stage c) and d). Furthermore, it allows to determine on small scale the protein expression characteristics by analysing the titer. Thereby, a highly efficient screening method is provided which has important advantages over existing systems in particular for use in industry.

Any polypeptide of interest can be expressed with the method of the present invention. Preferably, the polypeptide of interest is for use in medicine or diagnostics. Preferably, the polypeptide of interest is a pharmaceutically or therapeutically active polypeptide, or a research tool to be utilized in diagnostic or other assays and the like. A polypeptide is accordingly not limited to any particular protein or group of proteins, but may on the contrary be any protein, of any size, function or origin, which one desires to select and/or express by the methods described herein. Accordingly, several different polypeptides of interest may be expressed/produced. The term polypeptide refers to a molecule comprising a polymer of amino acids linked together by a peptide bond(s). Polypeptides include polypeptides of any length, including proteins (e.g. having more than 50 amino acids) and peptides (e.g. 2-49 amino acids). Polypeptides include proteins and/or peptides of any activity or bioactivity, including e.g. bioactive polypeptides such as enzymatic proteins or peptides (e.g. proteases, kinases, phosphatases), receptor proteins or peptides, transporter proteins or peptides, bactericidal and/or endotoxin-binding proteins, structural proteins or peptides, immune polypeptides, toxins, antibiotics, hormones, growth factors, vaccines or the like. Said polypeptide may be selected from the group consisting of peptide hormones, interleukins, tissue plasminogen activators, cytokines, immunoglobulins, in particular antibodies or functional antibody fragments or variants thereof and Fc-fusion proteins. According to one embodiment, the polypeptide of interest is glycosylated. The polypeptide of interest that is expressed according to the teachings described herein may also be a subunit or domain of one of the foregoing polypeptides, such as e.g. a heavy chain or a light chain of an antibody or a functional fragment or derivative thereof. In a preferred embodiment the polypeptide of interest is an immunoglobulin molecule, more preferably an antibody, or a subunit or domain thereof such as e.g. the heavy or light chain of an antibody or a single domain antibody. The term “antibody” as used herein particularly refers to a protein comprising at least two heavy chains and two light chains connected by disulfide bonds. The antibody can be a diagnostic antibody, or a pharmaceutically or therapeutically active antibody. The term “antibody” includes naturally occurring antibodies as well as all recombinant forms of antibodies, e.g., humanized antibodies, fully human antibodies and chimeric antibodies. Each heavy chain is usually comprised of a heavy chain variable region (VH) and a heavy chain constant region (CH). Each light chain is usually comprised of a light chain variable region (VL) and a light chain constant region (CL). The heavy chain-constant region comprises three or—in the case of antibodies of the IgM- or IgE-type—four heavy chain-constant domains (CH1, CH2, CH3 and CH4) wherein the first constant domain CH1 is adjacent to the variable region and may be connected to the second constant domain CH2 by a hinge region. The light chain-constant region consists only of one constant domain. The term “antibody”, however, also includes other types of antibodies such as heavy chain antibodies, i.e. antibodies only composed of one or more, in particular two heavy chains, and nanobodies, i.e. antibodies only composed of a single monomeric variable domain. As discussed above, the polynucleotide encoding the product of interest may also encode one or more subunits or domains of an antibody, e.g. a heavy or a light chain or a functional fragment or derivative, as polypeptide of interest. A “functional fragment or derivative” of an antibody in particular refers to a protein or glycoprotein which is derived from an antibody and is capable of binding to the same antigen, in particular to the same epitope as the antibody. The same applies mutatis mutandis for a fragment or derivative of an immunoglobulin molecule, a heavy chain or the light chain. It has been shown that the antigen-binding function of an antibody can be executed by fragments of a full-length antibody or derivatives thereof. Examples of fragments or derivatives of an antibody include (i) Fab fragments, monovalent fragments consisting of the variable region and the first constant domain of each the heavy and the light chain; (ii) F(ab)₂ fragments, bivalent fragments comprising two Fab fragments linked by a disulfide bridge at the hinge region; (iii) Fd fragments consisting of the variable region and the first constant domain CH1 of the heavy chain; (iv) Fv fragments consisting of the heavy chain and light chain variable region of a single arm of an antibody; (v) scFv fragments, Fv fragments consisting of a single polypeptide chain; (vi) (Fv)₂ fragments consisting of two Fv fragments covalently linked together; (vii) a heavy chain variable domain; and (viii) multibodies consisting of a heavy chain variable region and a light chain variable region covalently linked together in such a manner that association of the heavy chain and light chain variable regions can only occur intermolecular but not intramolecular. These antibody fragments and derivatives can be obtained using conventional techniques known to those with skill in the art.

The expression vector or the combination of at least two expression vectors used herein may additionally comprise further vector elements. E.g. at least one additional polynucleotide encoding a further polypeptide of interest can be comprised. As explained above and as becomes apparent from the above described examples of polypeptides that can be expressed according to the present teachings, the final polypeptide that is to be produced and secreted by the host cell can also be a dimeric or multimeric protein. A preferred example of a respective protein is an immunoglobulin molecule, in particular an antibody that comprises e.g. heavy and light chains. There are several options for producing a respective dimeric or multimeric protein and appropriate vector designs are known in the art. According to one embodiment, two or more subunits or domains of said dimeric or multimeric protein are expressed from one expression cassette. In this embodiment, one long transcript is obtained from the respective expression cassette that comprises the coding regions of the individual subunits or domains of the dimeric or multimeric protein. According to one embodiment, at least one IRES element (internal ribosomal entry site) is functionally located between the coding regions of the individual subunits or domains and each coding region is preceded by a secretory leader sequence. Thereby, it is ensured that separate translation products are obtained from said transcript and that the final dimeric or multimeric protein can be correctly assembled and secreted. Respective technologies are known in the prior art.

However, it is also within the scope and for some embodiments such as the expression of antibodies it is even preferred to express the individual subunits or domains of a dimeric or multimeric protein from different expression cassettes. According to one embodiment, the expression cassette used for expressing the product of interest is a monocistronic expression cassette. Preferably, all expression cassettes comprised in the vector or combination of vectors are monocistronic. According to one embodiment, accordingly, each expression cassette comprises a polynucleotide encoding one subunit or domain of the dimeric or multimeric protein as polypeptide of interest, e.g. one expression cassette encodes the light chain of an antibody, another expression cassette encodes the heavy chain of the antibody. After expression of the individual subunits/domains from the individual expression cassettes, the final dimeric or multimeric protein such as an antibody is assembled and secreted from the host cell.

This embodiment is particularly suitable for expressing immunoglobulin molecules such as antibodies. In this case, a first polynucleotide encoding a product of interest encodes e.g. the heavy or the light chain of an immunoglobulin molecule and a second polynucleotide encoding a product of interest encodes the other chain of the immunoglobulin chain. Further general vector elements that might be useful are known in the prior art and include but are not limited to origins of replication, further selectable markers or promoters for expression in different host cells.

According to one embodiment, the expression vector or combination of at least two expression vectors comprises at least one polynucleotide encoding the heavy chain of an immunoglobulin molecule or functional fragment thereof and at least one polynucleotide encoding the light chain of an immunoglobulin molecule or a functional fragment thereof. Said polynucleotides may be located on the same or on different expression vectors in case a combination of at least two expression vectors is used. Upon expression of said polynucleotides in the transfected host cell, a functional immunoglobulin molecule is obtained and preferably is secreted from the host cell. The polynucleotide encoding the heavy chain of an immunoglobulin molecule and the polynucleotide encoding the light chain of an immunoglobulin molecule may be comprised in the same expression cassette or in separate expression cassettes as is described briefly herein. Preferably, the expression cassettes described above, wherein a portion of the polypeptide of interest is produced as membrane-anchored fusion polypeptide by translational readthrough or alternative splicing, are used for expressing the antibody heavy chain.

The method according to the present invention renders a large number of cell clones, which have a high production rate and favourable growth characteristics. The chances are increased to identify a cell clone which not only produces the polypeptide of interest with high yield, but also have good and stable growth characteristics. Furthermore, it was found that clones that are selected with the method described herein show a very good expression stability.

As described, the cells that are selected according to the method described herein can then be subjected to a further selection stage subsequent to stage e). Therein, it can be for example determined/selected on a larger scale whether a cell not only has good growth characteristics but furthermore, also shows favourable growth characteristics and therefore is suitable for use on industrial scale. Clones which show the best performance on the small scale in the automated cell handling system and which are thus selected can then be cultivated out of the automated cell handling system and can be evaluated on a larger scale, e.g. in 24-well plate batches and shake flask screening formats.

The serial combination of FACS and clone picking as taught herein, significantly reduces the costs for screening. Approximately 50% of the costs can be saved thereby. Furthermore, one can look at more clones in order to identify clones having the desired characteristics (at the end 50% and more) or more projects can be handled in parallel, as the use of the capacity of the cloning robot is optimized, as only high producing cells showing good characteristics in the clone picking step are transferred and analysed in the automated cell handling system. Furthermore, even though in the present invention an additional selection stage is included, it was found that the overall screening time is not necessarily increased as is shown in the examples.

Also provided is a method for producing a polypeptide of interest, comprising
a) culturing a cell clone selected according to the method of the first aspect under conditions that allow for the expression of the polypeptide of interest; and
b) isolating the polypeptide of interest from the cell culture medium and/or from the cells.

The method has the advantage that the polypeptide of interest can be stably produced with a very high yield when performing the screening method according to the present disclosure for selecting appropriate host cells for expression. Thus, an improved method is provided for producing a polypeptide of interest. Suitable host cells are described above; we refer to the above disclosure.

The expressed product of interest may be obtained by disrupting the host cells. The polypeptides may also be expressed, e.g. secreted into the culture medium and can be obtained therefrom. For this purpose, an appropriate leader peptide is provided in the polypeptide of interest. Leader sequences and expression cassette designs to achieve secretion are well known in the prior art. Also a combination of the respective methods is possible. Thereby, polypeptides such as proteins can be produced and obtained/isolated efficiently with high yield. The obtained polypeptide of interest may also be subject to further processing steps such as e.g. purification and/or modification steps in order to produce the polypeptide of interest in the desired quality. According to one embodiment, said host cells are cultured under serum-free conditions.

The method for producing the polypeptide of interest may comprise at least one of the following steps:
isolating the polypeptide of interest from said cell culture medium and/or from said host cell; and/or
processing the isolated polypeptide of interest.

The polypeptide of interest that is produced may be recovered, further purified, isolated, processed and/or modified by methods known in the art. For example, the product may be recovered from the nutrient medium by conventional procedures including, but not limited to, centrifugation, filtration, ultra-filtration, extraction or precipitation. Further processing steps such as purification steps may be performed by a variety of procedures known in the art including, but not limited to, chromatography (e.g. ion exchange, affinity, hydrophobic, chromatofocusing, and size exclusion), electrophoretic procedures (e.g. preparative isoelectric focusing), differential solubility (e.g. ammonium sulfate precipitation) or extraction. Furthermore, the isolated and purified polypeptide of interest may be further processed such as formulated into a composition, e.g. a pharmaceutical composition.

Numeric ranges described herein are inclusive of the numbers defining the range. The headings provided herein are not limitations of the various aspects or embodiments of this disclosure which can be read by reference to the specification as a whole. According to one embodiment, subject-matter described herein as comprising certain elements also refers to subject-matter consisting of the respective elements. In particular, the polynucleotides described herein as comprising certain sequences may also consist of the respective sequences. It is preferred to select and combine preferred embodiments described herein and the specific subject-matter arising from a respective combination of preferred embodiments also belongs to the present disclosure.

The following examples serve to illustrate the present invention without in any way limiting the scope thereof. In particular, the examples relate to preferred embodiments of the present invention.

All documents cited herein are incorporated by reference.

### EXAMPLES

In general, the suitable materials, such as reagents, are familiar to the skilled person, commercially available and can be used in accordance with the manufacturer's instructions. The examples are performed according to the described instructions.

### Example 1

### Combinatorial Cloning Using Both Fluorescence Activated Cell Sorting and Colony Picking with the ClonePix FL

### Material and Methods

### 1.1 Vectors

In the examples, two types of expression vectors were used. Both expression vectors expressed as polypeptide of interest an IgG antibody and comprise the neo gene and the DHFR gene as selectable marker. One expression vector was a specific FACS vector (FACS vector) wherein the expression cassette comprising the polynucleotide encoding the polypeptide of interest has a design that upon stop codon read through a fusion polypeptide comprising the antibody fused to a transmembrane anchor is obtained which is displayed on the expressing host cell. The predominant amount of polypeptide of interest is expressed as secreted polypeptide of interest. A respective vector is described in WO 2010/022961. The other vector was a standard expression vector (standard vector) wherein the expression cassette comprising the polynucleotide encoding the polypeptide of interest does not comprise a respective membrane anchor so that the antibody is only produced in its secreted form. A respective vector is described in WO 2009/080720.

### 1.2. Transfection

CHO cells were used as model cells. CHO cells are the standard when producing polypeptides in particular pharmaceutical polypeptides. Therefore, CHO cells were used as model cells. The principles shown in the examples, however, also apply to other mammalian cell lines. A single vial containing the parental CHO cell line was cultivated in proprietary medium. The cells were passaged two to three times per week into fresh media and were maintained in exponential growth phase. The parental CHO cells with a viability higher than 90% were used for the transfection performed by nucleofection method in order to introduce the expression vectors. Depending on the cell viability, geneticin (G418) selection was started 24 to 48 hours after electroporation by adding geneticin containing selective medium to the cells. Once the cells recovered to the first selection step (viability higher that 80%), they were subsequently submitted to the second selection step. The cells were then passaged in a medium supplemented with methotrexate (MTX) and free of geneticin. This second pre-selection step was thus based on the DHFR/MTX system. It favours the selection of cells that have integrated the expression vector at a locus favourable to reach high expression levels and the isolation of cells that express the polypeptide of interest with high yield. The cells which recovered from the second selection step in MTX containing medium, were called amplified pools. The individual amplified pools were cryo-preserved to be used further on for the cloning phase.

### 1.3 Labelling of Cells for Fluorescence Activated Cell Sorting

When the amplified pool reached a cell density of 1×10⁷ cells/ml, a total of 3×10⁸ cells were prepared for labeling. First, the cells were centrifuged and washed with 5 ml of chilled PBS. After a second centrifugation the cells were resuspended in 1 ml of cold PBS and incubated on ice for 30 minutes in the dark with 225 μl of FITC-conjugated anti-IgG antibody as detection compound. This detection compound can bind to the fusion polypeptide that is displayed in the FACS vector based system on the cell surface, namely the IgG polypeptide of interest. After incubation, the cells were washed twice with 5 ml of cold PBS and finally resuspended in 3 ml of PBS for fluorescence activated cell sorting.

### 1.4 Fluorescence Activated Cell Sorting (FACS)

The enrichment of the amplified cell pools was performed with a FACSAria (Becton Dickinson) equipped with an Automated Cell Deposition Unit (ACDU) using FACSDiva software. A low powered air-cooled and a solid-state laser (Coherent® Sapphire™ solid state) tuned to 488 nm was used to excite fluorescein dyes bound to the secondary antibody used as label. The relative FITC fluorescence intensity was measured on E detector through a 530/30 BP filter. Five percent of the highest FITC fluorescent cells were gated, sorted in bulk and collected in 6 well plates containing proprietary medium supplemented with gentamycin to prevent contaminations.

### 1.5 Plating of Flow Cytometry-Enriched Pools in Semi-Solid Media

Once the amplified pools were enriched by flow cytometry, the cells were immediately transferred to culture plates with semi-solid medium containing 50% of 2× concentrated medium supplemented with feed and 50% of CloneMatrix. To ensure a correct density of colonies, the cells were seeded at 200 cells/ml. The semi-solid medium was additionally supplemented with 20 μl of FITC-conjugated affiniPure F(ab′)₂ fragment anti-human IgG for the labeling of secreting colonies. After 11 to 14 days of cultivation, the colonies were ready for picking.

1.6 Automated Colony Picking with ClonePix FL Robot

Prior to any picking, the different elements of the ClonePix FL (head, pins, ClonePix FL cabinet) were properly cleaned to prevent contaminations. Stringent picking parameters were setup to prevent cross-contaminations and ensure cell clonality. The following parameters were used for selection: average colony diameter (picking at day 8 to 12) 0.32 mm; edge excluded; too big: total area ≦0.7 mm; too small: total area ≦0.1 mm; irregular 1: compactness ≦0.6 mm; irregular 2: Axio ratio—0.6 mm; proximity ≧1 mm. For the fluorescence Exterior median intensity was used.

The picked colonies were transferred to 96-well culture plates and placed in the incubator of an automated cloning robot for further handling.

### 1.7 Clone Processing

The 96-well plates containing the picked colonies were transferred to the incubator of an automated cell handling system (cloning robot). The clones were regularly controlled for growth by a cell confluence imager and then diluted by the liquid handling system of the robot according to the ongoing program. The productivity performance of each individual clone was then evaluated by 96-well plate batches and a titer ranking was made to select the best producing ones. Those clones which showed the best performance were cultivated out of the cloning robot and evaluated in 24-well plate batches and shake flask screening formats.

### 1.8 Determination of Clone Productivity

a) Batch cultures were performed in agitated 24-well plates with a working volume of 1 ml. After 10 days of cultivation, culture supernatants were taken and antibody titers measured by protein-A HPLC.
b) The fed-batch cultures were performed in 250 ml shake flask with 100 ml working volume and cultivated in a shaker cabinet (not humidified) at 150 rpm and 10% CO₂. The fed-batches were initiated with a starting cell density of 1×10⁵ cells/ml and a viability greater than 90%. After 13 days of cultivation, culture supernatants were taken and antibody titers measured by protein-A HPLC.

### Results

Once the amplified pools were enriched by selection of the highest fluorescent cells with the flow cytometer, they were immediately plated in semi-solid media and incubated at 37° C. and 10% CO₂. After 10 to 14 days, the colonies were picked by the ClonePix FL according to their fluorescence intensities and transferred to 96 well plates to be further incubated in the automated handling system (cloning robot).

Tables 1 and 2 show the antibody titer correlation between the 24-well plate batches and the fed-batches in 250 ml shake flask according to the FITC intensity of the picked colonies.

The data shows that the picking of the middle and the high FITC fluorescent intensity colonies enables to isolate the highest producing clones either by using the standard or the FACS vector constructs. Higher productivities were achieved with the FACS vector. These results are strongly emphasized and further supported by the good correlation obtained between the 24-well plate batches and the shake flask fed-batches. This demonstrates that the high expression rates of the clones obtained by the selection method are maintained in larger cultures and hence, are stable during up-scaling.

TABLE 1
Results obtained with the standard vector (1 μM MTX).
Shown is the titer in mg/L

| | 24 well plate | Shake flask |
|---|---|---|
| | batches | screening II |
| Clones derived | 249 | 552 |
| from picking of | 64 | 60 |
| low fluorescent | 415 | 592 |
| colonies | 4 | 6 |
| | 92 | 339 |
| | 401 | 547 |
| | 437 | 391 |
| Clones derived | 462 | 813 |
| from picking of | 439 | 1430 |
| middle | 786 | 1846 |
| fluorescent | 295 | 518 |
| colonies | 170 | 242 |
| Clones derived | 32 | 352 |
| from picking of | 177 | 443 |
| high fluorescent | 877 | 1818 |
| colonies | 1107 | 2294 |
| | 996 | 2450 |
| | 17 | 24 |

TABLE 2
Results obtained with the FACS vector (500 nM MTX).
Shown is the titer in mg/L

| | 24 well plate | Shake flask |
|---|---|---|
| | batches | screening II |
| Clones derived | 255 | 533 |
| from picking of | 263 | 818 |
| low fluorescent | 763 | 1996 |
| colonies | | |
| Clones derived | 225 | 1055 |
| from picking of | 699 | 2773 |
| middle | 460 | 1888 |
| fluorescent | 222 | 638 |
| colonies | 505 | 1038 |
| | 239 | 262 |
| | 571 | 1413 |
| | 304 | 819 |
| | 1376 | 3380 |
| Clones derived | 269 | 1313 |
| from picking of | 256 | 836 |
| high fluorescent | 221 | 1023 |
| colonies | 214 | 1189 |
| | 234 | 987 |
| | 303 | 2231 |
| | 477 | 1569 |
| | 284 | 1178 |
| | 826 | 980 |
| | 823 | 2477 |
| | 219 | 773 |
| | 401 | 1265 |
| | 244 | 838 |
| | 216 | 807 |
| | 280 | 1368 |
| | 408 | 1094 |
| | 745 | 1999 |
| | 789 | 2588 |
| | 315 | 1068 |
| | 319 | 1388 |

### Example 2

### Comparison of Cloning Efficiencies—Fluorescence Activated Cell Sorting and Colony Picking with the ClonePix FL Automated Clone Picking Apparatus

Five different cell cloning projects using either fluorescence activated cell sorting or ClonePix FL have been analyzed in respect to the obtained cloning efficiency. The data have shown that with the CHO cells an average of 30% cloning efficiency can be achieved using the flow cytometer. When the cloning step is performed by the ClonePix FL, an average of 73% of cloning efficiency can be observed. Therefore, the percentage of growing clones by using the ClonePix FL is increased in average by a factor of 2.5 compared to the results expected by flow cytometry assisted cloning. For the same amount of 96 well plate transferred, the number of clone handled by the cloning robot is increased by a factor of 2.5, which enables the robot to almost reaching its maximal degree of work efficiency.

### Example 3

### Timelines—Clone Selection Using Both FACS and Clone Picking Requires Approximately the Same Amount of Time as Clone Selection by FACS Alone

For the integration of the ClonePix FL in a FACS based clone selection CHO platform, the duration of the following two different processes have been compared:
1. fluorescence activated cell sorting+clone propagation using the cloning robot
2. fluorescence activated cell sorting, colony picking with ClonePix FL+clone propagation using the cloning robot

The experiments showed that the cloning process made with FACS cloning+cloning robot and the one made with FACS enrichment+ClonePix FL+cloning robot have similar duration. Although the process including the ClonePix-FL contains one additional step, the global duration is equivalent because the cell recovery after ClonePix FL picking is much better compared to the cell recovery after FACS cloning. Therefore, by integrating the colony picking step into the selection process as described herein, results in that more clones having the desired characteristics with respect to yield and growth characteristics can be obtained.

1. A screening method for selecting at least one cell clone with desired colony characteristics expressing a polypeptide of interest, the method comprising
a) providing a plurality of eukaryotic host cells comprising a heterologous nucleic acid comprising a polynucleotide encoding the polypeptide of interest;
b) cultivating the eukaryotic host cells;
c) performing a first, flow cytometry based selection, comprising
selecting a plurality of eukaryotic host cells expressing the polypeptide of interest with desired yield using flow cytometry;
d) performing a second, colony picking based selection, comprising
obtaining single cell colonies in a medium which prevents the migration of the cells from a plurality of eukaryotic host cells selected in stage c);
detecting within the obtained cell colonies one or more cell colonies having desired colony characteristics;
picking one or more cell colonies having the desired colony characteristics;
e) cultivating the picked cell colonies to provide cell clones.

2. The method according to claim 1, wherein in stage e) the picked colonies are cultivated in an incubator of an automated cell handling system.

3. The method according to claim 2, wherein stage e) comprises selecting cell clones for their productivity performance.

4. The method according to claim 1, wherein stage c) comprises selecting a plurality of eukaryotic host cells expressing the polypeptide of interest with a desired yield based upon to the presence or amount of the polypeptide of interest using flow cytometry.

5. The method according to claim 1, wherein
in stage a), the plurality of eukaryotic host cells provided comprise a heterologous expression cassette comprising
i) the polynucleotide encoding the polypeptide of interest,
ii) at least one stop codon downstream of the polynucleotide encoding the polypeptide of interest, and
iii) a further polynucleotide downstream of the stop codon encoding a membrane anchor and/or a signal for a membrane anchor; and
in stage b) cultivating the eukaryotic host cells is performed to allow expression of the polypeptide of interest wherein at least a portion of the polypeptide of interest is expressed as fusion polypeptide comprising a membrane anchor, wherein said fusion polypeptide is displayed on the surface of said host cell; and
in stage c), the first, flow cytometry based selection comprises
selecting a plurality of eukaryotic host cells expressing the polypeptide of interest with a desired yield based upon the presence or amount of the fusion polypeptide displayed on the cell surface using flow cytometry.

6. The method according to claim 1, wherein
in stage a), the plurality of eukaryotic host cells provided comprise a heterologous expression cassette comprising
i) the polynucleotide encoding the polypeptide of interest,
ii) an intron comprising a 5′ splice donor site and a 3′ splice acceptor site and comprising an in frame translational stop codon and a polyadenylation signal and
iii) a polynucleotide downstream of said intron encoding a membrane anchor and/or a signal for a membrane anchor; and
in stage b) cultivating the eukaryotic host cells is performed to allow expression of the polypeptide of interest wherein at least a portion of the polypeptide of interest is expressed as fusion polypeptide comprising a membrane anchor, wherein said fusion polypeptide is displayed on the surface of said host cell; and
in stage c) the first, flow cytometry based selection comprises
selecting a plurality of eukaryotic host cells expressing the polypeptide of interest with a desired yield based upon the presence or amount of the fusion polypeptide displayed on the cell surface using flow cytometry.

7. The method according to claim 5, wherein stage c) comprises selecting a plurality of eukaryotic host cells expressing the polypeptide of interest with a desired yield based upon the presence or amount of the fusion polypeptide displayed on the cell surface using flow cytometry by contacting the eukaryotic host cells with a detection compound binding the fusion polypeptide displayed on the cell surface and selecting a plurality of eukaryotic host cells expressing the polypeptide of interest with a desired yield based upon the presence or amount of the bound detection compound using flow cytometry.

8. The method according to claim 1, wherein in stage c) producing cells, preferably the highest producing cells, comprised in the cell culture are selected and sorted into a cell pool using FACS and wherein at least a portion of said FACS selected cell pool comprising high producing cells is used in stage d) for obtaining cell colonies.

9. The method according to claim 1, wherein the medium which prevents the migration of the cells used in stage d) is a semi-solid medium or a solid medium.

10. The method according to claim 1, wherein in stage d) the colony characteristics are selected from the group consisting of expression of the polypeptide of interest, in particular the expression yield, characteristics of the cell colony, cell colony growth characteristics, in particular the colony size, the colony shape, the size and/or shape of the cells in the colony.

11. The method according to claim 1, wherein in stage d) cell colonies are picked based on the expression yield and optionally the colony size and/or colony shape.

12. The method according to claim 1, wherein stage d) comprises
obtaining single cell colonies in a medium which prevents the migration of the cells from a plurality of eukaryotic host cells selected in stage c), wherein the host cells are allowed to express the polypeptide of interest, and wherein at least a portion of the polypeptide of interest is expressed as fusion polypeptide comprising a membrane anchor, wherein said fusion polypeptide is displayed on the surface of the host cells;
detecting within the obtained cell colonies one or more cell colonies having desired colony characteristics, preferably including determining the presence and/or amount of the polypeptide of interest in the area of the cell colonies;
picking one or more cell colonies having the desired colony characteristics.

13. The method according to claim 1, wherein stage d) comprises
obtaining single cell colonies in a medium which prevents the migration of the cells from a plurality of eukaryotic host cells selected in stage c), wherein the host cells are allowed to express the polypeptide of interest, and wherein the polypeptide of interest is secreted by the host cells;
detecting within the obtained cell colonies one or more cell colonies having desired colony characteristics, preferably including determining the presence and/or amount of the polypeptide of interest in the area surrounding the cell colonies;
picking one or more cell colonies having the desired colony characteristics.

14. The method according to claim 13, wherein stage d) comprises the use of a detection compound capable of associating with the polypeptide of interest for determining the presence and/or amount of the polypeptide of interest.

15. The method according to claim 1, wherein stage e) comprises one or more of the following:
i) a sample of the obtained cell culture is removed for determining the amount of expressed polypeptide of interest;
ii) the cell growth of the picked colonies is determined; and/or
iii) one or more daughter cultures are prepared from the parent culture that is obtained from cultivating the picked colonies.

16. The method according to claim 1, wherein the eukaryotic host cell is a mammalian host cell, preferably a CHO cell.

17. The method according to claim 1, wherein the heterologous nucleic acid comprising a polynucleotide encoding the polypeptide of interest is stably introduced into the genome of the host cell.

18. The method according to claim 1, wherein the heterologous nucleic acid is an expression vector which additionally comprises at least one polynucleotide encoding a selectable marker and wherein in stage b) the host cells are cultivated under conditions providing a corresponding selection pressure to identify successfully transfected cells.

19. A method for producing a polypeptide of interest, comprising
a) culturing a cell clone selected according to the method of claim 1 under conditions that allow for the expression of the polypeptide of interest; and
b) isolating the polypeptide of interest from the cell culture medium and/or from the cells.

20. The method according to claim 19, further comprising
c) processing the isolated polypeptide of interest.
